(19) United States
(12) Patent Application Publication
(10) Pub. No.: US 2026/0153469 A1
(43) Pub. Date: Jun. 4, 2026

(54) THERMAL CONDUCTIVITY DETECTION UNIT AND GAS CHROMATOGRAPH

(71) Applicant: SHIMADZU CORPORATION, Kyoto-shi (JP)

(72) Inventor: Yosuke KOGA, Kyoto-shi (JP)

(21) Appl. No.: 19/403,589

(22) Filed: Nov. 28, 2025

(30) Foreign Application Priority Data
Nov. 29, 2024 (JP) 2024-208892

(51) Int. Cl. G01N 27/18; G01N 33/00

(52) U.S. Cl. CPC G01N 27/18; G01N 33/0073

(57) ABSTRACT

A controller that controls a thermal conductivity detector is configured to control an output of a heating device of the thermal conductivity detector with an output corresponding to a measurement result of a temperature sensor in a basic control. However, the controller is configured to, when a temperature of a first member housing a filament and the temperature sensor is outside a first range with respect to a temperature of a second member housing the heating device, control the output of the heating device with an output corresponding to the measurement result in an output reduction control. In the output reduction control, the output of the heating device is controlled with an output that is reduced from the output corresponding to the measurement result in the basic control.

### TECHNICAL FIELD

The present invention relates to improving the accuracy of operation control of a heating device of a thermal conductivity detector.

### BACKGROUND ART

Conventionally, as described in, for example, Japanese Unexamined Patent Application Publication No. 2020-041989 (Patent Literature 1), a thermal conductivity detector has been provided with a heating device (heater). This has minimized changes in the temperature of the portion that detects thermal conductivity, including the filament, due to factors other than the composition or concentration of the gas to be detected.

### CITATION LIST

### Patent Literature

[Patent Literature 1] Japanese Unexamined Patent Application Publication No. 2020-041989

### SUMMARY OF INVENTION

### Technical Problem

In conventional thermal conductivity detectors, from the viewpoint of reducing manufacturing costs, a general-purpose unit housed in a member with relatively high thermal conductivity has sometimes been adopted as the heating device. In such a case, the filament is housed in a member with relatively low thermal conductivity, along with a temperature sensor. In such a case, if the operation of the heating device is controlled using the temperature detected by the temperature sensor, the member housing the heating device may reach a target temperature before the detected temperature reaches the target temperature. As a result, when the detected temperature reaches the target temperature, the temperature of the member housing the heating device may exceed the target temperature. Therefore, if the heating device is controlled based on the detected temperature, a temperature overshoot may occur. This causes a situation where a long time is required for the temperature of the said portion in the thermal conductivity detector to stabilize during analysis using the thermal conductivity detector, and there has been a demand for improved accuracy in the operation control of the heating device in the thermal conductivity detector.

The present invention has been conceived in view of such circumstances, and an object thereof is to provide a technology for improving the accuracy of operation control of a heating device of a thermal conductivity detector.

### Solution to Problem

A thermal conductivity detection unit according to an aspect of the present disclosure includes a thermal conductivity detector and a controller configured to control the thermal conductivity detector, wherein the thermal conductivity detector includes a first member, a second member having a higher thermal conductivity than the first member, a heating device housed in the second member, and a filament and a temperature sensor housed in the first member, and the controller is configured to control an output of the heating device with an output corresponding to a measurement result of the temperature sensor in a basic control, and when a temperature of the first member is outside a first range with respect to a temperature of the second member, control the output of the heating device with an output corresponding to the measurement result in an output reduction control, wherein in the output reduction control, the output of the heating device is controlled with an output that is reduced from the output corresponding to the measurement result in the basic control.

A gas chromatograph according to an aspect of the present disclosure includes a sample vaporization unit that generates a sample gas by vaporizing a sample, a column that separates components of the sample gas generated by the sample vaporization unit, and the above-described thermal conductivity detection unit, wherein the thermal conductivity detection unit detects the thermal conductivity of the sample gas for each component separated by the column.

### Advantageous Effects of Invention

According to an aspect of the present disclosure, a technology is provided for improving the accuracy of operation control of a heating device of a thermal conductivity detector.

### BRIEF DESCRIPTION OF DRAWINGS

FIG. 1 is a block diagram showing a configuration of a gas chromatograph including an embodiment of a thermal conductivity detection unit.

FIG. 2 is a diagram showing an example of a control block related to the operation control of the heating device.

FIG. 3 is a diagram showing an example of information used for setting a threshold value.

FIG. 4 is a diagram showing the information shown in FIG. 3 with different annotations from FIG. 3.

FIG. 5 is a diagram showing the information shown in FIG. 3 with different annotations from FIG. 3.

FIG. 6 is a flowchart of an output control process of a heating device 70H for controlling a first conduit 71 housing a filament F to a target temperature.

### DESCRIPTION OF EMBODIMENTS

Hereinafter, embodiments of the present disclosure will be described in detail with reference to the drawings. In the drawings, the same or corresponding parts are denoted by the same reference numerals, and a description thereof will not be repeated.

### Schematic Configuration and Basic Operation of Gas Chromatograph

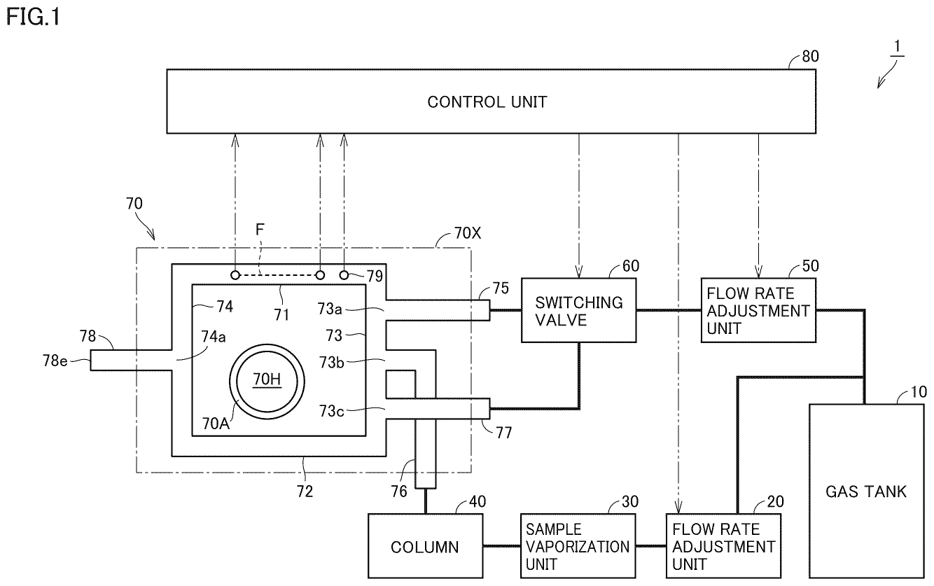

FIG. 1 is a block diagram showing a configuration of a gas chromatograph including an embodiment of a thermal conductivity detection unit. As shown in FIG. 1, a gas chromatograph 1 includes a gas tank 10, a flow rate adjustment unit 20, a sample vaporization unit 30, a column 40, a flow rate adjustment unit 50, a switching valve 60, a thermal conductivity detector 70, and a control unit 80.

The gas tank 10 stores a carrier gas for guiding a sample gas to the column 40 and the thermal conductivity detector 70. As the carrier gas, for example, an inert gas such as helium gas is used.

The gas tank 10 supplies the carrier gas to two flow rate adjustment units 20 and 50 via a branch conduit. One flow rate adjustment unit 20 supplies the carrier gas at a predetermined flow rate to the sample vaporization unit 30 based on the control of the control unit 80.

The sample vaporization unit 30 includes an injector and a vaporization chamber. A sample is injected into the vaporization chamber of the sample vaporization unit 30 via the injector. The internal atmosphere of the vaporization chamber is maintained in a state where the sample vaporizes. Thereby, the sample injected into the vaporization chamber is vaporized therein. The sample vaporization unit 30 supplies the vaporized sample to the column 40 while mixing it with the carrier gas supplied from the flow rate adjustment unit 20. In the following description, the gas containing the components of the sample vaporized in the sample vaporization unit 30 is collectively referred to as a sample gas.

The column 40 is housed in a column oven (not shown). In the column 40, each component of the sample gas supplied from the sample vaporization unit 30 is separated. The column 40 supplies the sample gas, separated for each component, to a sample introduction conduit 76 of the thermal conductivity detector 70.

The flow rate adjustment unit 50 supplies a carrier gas at a predetermined flow rate to the switching valve 60 based on the control of the control unit 80. The switching valve 60 is, for example, a three-way solenoid valve, and is connected to the flow rate adjustment unit 50 and also to two later-described carrier gas introduction conduits 75 and 77 of the thermal conductivity detector 70. The flow rate adjustment unit 50 supplies the carrier gas supplied from the flow rate adjustment unit 50 to either one of the carrier gas introduction conduits 75 and 77 based on the control of the control unit 80.

Note that as a configuration for supplying the carrier gas passing through the flow rate adjustment unit 50 to either one of the two carrier gas introduction conduits 75 and 77, a switching mechanism including a plurality of control valves and a branch conduit may be used instead of the switching valve 60. For example, a main conduit is connected to the flow rate adjustment unit 50, and two sub-conduits are respectively connected to the carrier gas introduction conduits 75 and 77. Also, two control valves are provided in the two sub-conduits, respectively. In this case, by controlling the open/closed state of the two control valves, the carrier gas supplied from the flow rate adjustment unit 50 can be selectively supplied to either one of the two carrier gas introduction conduits 75 and 77 of the thermal conductivity detector 70.

The thermal conductivity detector 70 according to the present embodiment includes a first conduit 71, a second conduit 72, a third conduit 73, a fourth conduit 74, carrier gas introduction conduits 75 and 77, a sample introduction conduit 76, and an exhaust conduit 78, each extending linearly. These plurality of conduits are formed, for example, by metal piping. Among the plurality of conduits of the thermal conductivity detector 70, the first to fourth conduits 71 to 74 are housed in a cell block 70X together with a heating device 70H. The cell block 70X is manufactured by processing and joining a plurality of metal plate-like members. In the cell block 70X, the heating device 70H is housed in an aluminum block 70A.

The first conduit 71 and the second conduit 72 are formed to face each other and extend in parallel. The third conduit 73 is formed to connect one end of the first conduit 71 and one end of the second conduit 72, and the fourth conduit 74 is formed to connect the other end of the first conduit 71 and the other end of the second conduit 72. A filament F is housed inside the first conduit 71. On the other hand, the filament F is not housed inside the second conduit 72. Note that a temperature sensor 79 is further housed inside the first conduit 71.

The third conduit 73 is provided with a first gas introduction part 73a, a second gas introduction part 73b, and a third gas introduction part 73c, arranged in this order. Among the first to third gas introduction parts 73a to 73c, the first gas introduction part 73a is closest to the first conduit 71, and the third gas introduction part 73c is closest to the third conduit 73.

The carrier gas introduction conduit 75 is formed to extend from the first gas introduction part 73a to the outside of the cell block 70X. The sample introduction conduit 76 is formed to extend from the second gas introduction part 73b to the outside of the cell block 70X. The carrier gas introduction conduit 77 is formed to extend from the third gas introduction part 73c to the outside of the cell block 70X.

The fourth conduit 74 is provided with a gas outlet part 74a. The exhaust conduit 78 is formed to extend from the gas outlet part 74a to the outside of the cell block 70X. A through-hole is formed in the gas outlet part 74a. Thereby, the internal space of the fourth conduit 74 and the internal space of the exhaust conduit 78 communicate with each other. The exhaust conduit 78 has an exhaust port 78e outside the cell block 70X.

The heating device 70H is controlled by the control unit 80 and maintains the space inside the cell block 70X at a temperature comparable to the temperature inside the vaporization chamber of the sample vaporization unit 30 or the temperature inside the column oven housing the column 40. As the heating device 70H, for example, a cartridge heater is used.

The control unit 80 is constituted by, for example, a CPU (Central Processing Unit) and a memory, or a microcomputer, and controls the operation of each component of the gas chromatograph 1 as described above. Further, the control unit 80 of this example further includes a drive circuit for driving the filament F and a detection circuit for detecting a change in the resistance of the filament F.

The above-mentioned switching valve 60 is switched between a first state of supplying the carrier gas to one carrier gas introduction conduit 75 at a predetermined cycle (e.g., about 100 msec) and a second state of supplying the carrier gas to the other carrier gas introduction conduit 77.

In this case, inside the third conduit 73 of the thermal conductivity detector 70, when the switching valve 60 is in the first state, the pressure in the space on the side of the first gas introduction part 73a becomes higher than that of the second gas introduction part 73b. Thereby, the sample gas supplied to the sample introduction conduit 76 flows through the second conduit 72 together with a part of the carrier gas introduced from the first gas introduction part 73a. The rest of the carrier gas introduced from the first gas introduction part 73a flows through the first conduit 71 as a reference gas.

On the other hand, inside the third conduit 73 of the thermal conductivity detector 70, when the switching valve 60 is in the second state, the pressure in the space on the side of the third gas introduction part 73c becomes higher than that of the second gas introduction part 73b. Thereby, the sample gas supplied to the sample introduction conduit 76 flows through the first conduit 71 together with a part of the carrier gas introduced from the third gas introduction part 73c. The rest of the carrier gas introduced from the third gas introduction part 73c flows through the second conduit 72.

Thereby, in the control unit 80, the thermal conductivity of the sample gas is measured based on the change in the resistance value of the filament F between when the reference gas passes around the filament F and when the sample gas passes around the filament F.

### Overview of Operation Control of Heating Device

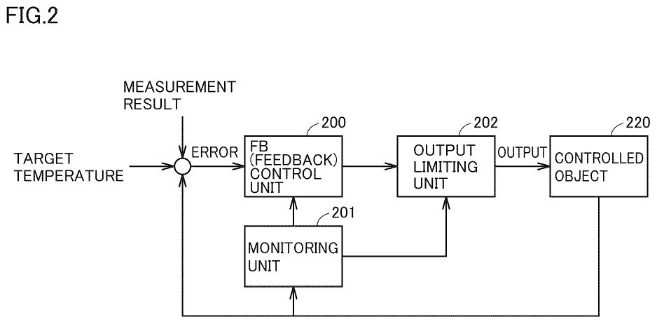

FIG. 2 is a diagram showing an example of a control block related to the operation control of the heating device. The example in FIG. 2 represents feedback (FB) control using the difference (error) between the measurement result of the temperature sensor 79 (the temperature measured by the temperature sensor 79) and a target temperature. FIG. 2 shows an FB control unit 200, a monitoring unit 201, an output limiting unit 202, and a controlled object 220. The controlled object 220 means the heating device 70H in FIG. 1.

Basically, the FB control unit 200 controls the output of the controlled object 220 according to a basic control. In the basic control, the output (value) of the controlled object 220 is set based on the above-mentioned error, and the controlled object 220 is controlled to achieve the set output.

In the example of FIG. 2, the monitoring unit 201 determines whether the temperature of the member housing the filament F and the temperature sensor 79 (the first conduit 71: the first member) is outside a first range with respect to the temperature of the member housing the heating device (the aluminum block 70A: the second member), and also determines whether it is within a second range.

Whether the temperature of the first member is outside the first range with respect to the temperature of the second member, and whether the temperature of the first member is within the second range with respect to the temperature of the second member, may be determined based on the direct measurement results of both temperatures, or may be determined indirectly by other methods.

When the monitoring unit 201 determines that the temperature of the first member is outside the first range with respect to the temperature of the second member, it instructs the output limiting unit 202 to execute an output reduction control. The output reduction control is a control for reducing the output instructed to be realized by the controlled object 220 to be lower than the output set by the FB control unit 200. The output controlled by the output limiting unit 202 may be a value obtained by subtracting a constant value from the output set by the FB control unit 200, or may be “zero”, as long as it is lower than the output set by the FB control unit 200.

The output limiting unit 202 executes the output reduction control in response to the instruction from the monitoring unit 201. Thereby, in the output reduction control, the output instructed to be realized by the controlled object 220 becomes lower than the output set by the FB control unit 200.

Thereafter, when the monitoring unit 201 determines that the temperature of the first member is within the second range with respect to the temperature of the second member, it instructs the output limiting unit 202 to release the output reduction control. The control of the controlled object 220 returns to the basic control. Thereby, the output instructed to the controlled object 220 returns to the output set by the FB control unit 200.

In the present embodiment, an example of the “basic control” is PID (Proportional Integral Differential) control. The basic control, as long as it is a control that uses the measurement result of the temperature sensor 79, may be a control that switches on/off the output of the controlled object 220 according to the measurement result, or a control in which the output of the controlled object 220 is set as a linear function of the measurement result.

In the present embodiment, the controlled object 220 (heating device) is housed in a member with a relatively high thermal conductivity. This allows the heat from the heating device to be efficiently propagated to other elements in the thermal conductivity detector. In this specification, aluminum (aluminum block) is shown as an example of a member with a relatively high thermal conductivity, but it is not limited to this, and other types of members such as copper may be used.

Further, the filament is housed in a member with a relatively low thermal conductivity. This makes it possible to stabilize the temperature of the filament and its surroundings. In this specification, stainless steel is shown as an example of a member with a relatively low thermal conductivity, but it is not limited to this, and other types of members such as titanium may be used.

Furthermore, the temperature sensor is housed in the same member as the filament, and the operation control of the heating device is performed based on the measurement result of the temperature sensor and in accordance with the basic control. This allows the temperature around the filament to be more reliably reflected in the measurement result of the temperature sensor and, in turn, more reliably reflected in the operation control of the heating device.

Then, in the operation control of the heating device, when the temperature of the heating device rises and the temperature of the member housing the heating device (the second member) deviates significantly from the temperature of the member housing the filament and the temperature sensor (the first member) (when the temperature difference between them is outside the first range), the output of the heating device is temporarily adjusted to be lower than the output corresponding to the measurement result in the basic control. This suppresses the occurrence of overshoot.

Thereafter, when the temperature of the member housing the filament and the temperature sensor (the first member) approaches the temperature of the member housing the heating device (the second member) (when the temperature difference between them is within the second range), the control of the output of the heating device is returned to the above-mentioned basic control. This allows the heating device to be controlled so that the temperature of the filament reaches the target temperature earlier.

As described above, according to the present disclosure, the operation of the heating device is controlled to make the temperature of the filament reach the target temperature earlier while suppressing the occurrence of overshoot.

### Premise for Threshold Setting

In the present embodiment, whether the temperature of the first member (the first conduit 71) is outside a first range with respect to the temperature of the second member (the aluminum block 70A) and whether the temperature of the first member is within a second range with respect to the temperature of the second member are determined using a threshold value.

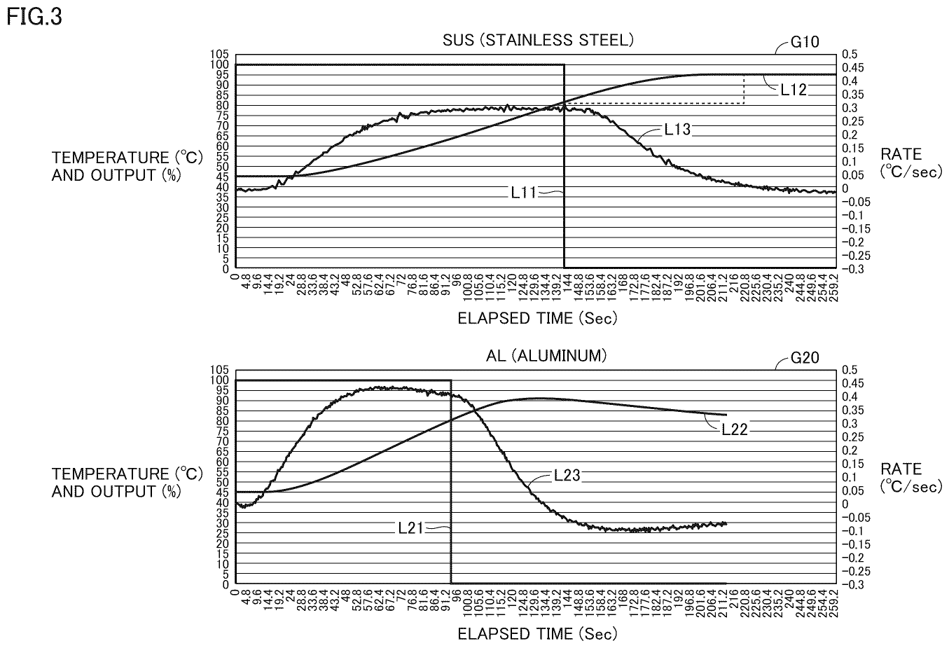

FIG. 3 is a diagram showing an example of information used for setting a threshold value. FIG. 3 shows a graph G10 and a graph G20 obtained in a gas chromatograph 1 modified for threshold setting. The modification is the addition of a temperature sensor (hereinafter also referred to as an “additional sensor”) attached to the aluminum block 70A.

Graph G10 shows the result of on/off control of the heating device 70H using the temperature sensor 79 and a target temperature (e.g., 80° C.) (controlling the output of the heating device 70H to 100% if the measurement result of the temperature sensor 79 is equal to or lower than the target temperature, and turning off the heating device 70H (output to 0%) if the measurement result exceeds the target temperature). In graph G10, changes in the output (%) of the heating device 70H (line L11), the measurement result (° C.) of the temperature sensor 79 (line L12), and the rate of the measurement result (° C./sec) (line L13) with the elapsed time from the start of heating by the heating device 70H are shown. The vertical axis for lines L11 and L12 is shown on the right side. The vertical axis for line L13 is shown on the left side.

Graph G20 shows the result of on/off control of the heating device 70H using the above-mentioned additional sensor and the target temperature (controlling the output of the heating device 70H to 100% if the temperature measured by the additional sensor is equal to or lower than the target temperature, and turning off the heating device 70H (output to 0%) if the measurement result exceeds the target temperature). In graph G20, changes in the output (%) of the heating device 70H (line L21), the measurement result (° C.) of the temperature sensor 79 (line L22), and the rate of the measurement result (° C./sec) (line L23) with the elapsed time from the start of heating by the heating device 70H are shown. The vertical axis for lines L21 and L22 is shown on the right side. The vertical axis for line L23 is shown on the left side.

In graph G10, line L11 indicates that after the output of the heating device 70H was turned on at 100%, the output was switched off at 14 seconds. Thus, the rise time from the start of heating in the first member is 14 seconds.

Further, in graph G10, line L11, as indicated by the dashed line, shows that the measurement result of the temperature sensor 79 increases by 15° C. after the output of the heating device 70H is switched off.

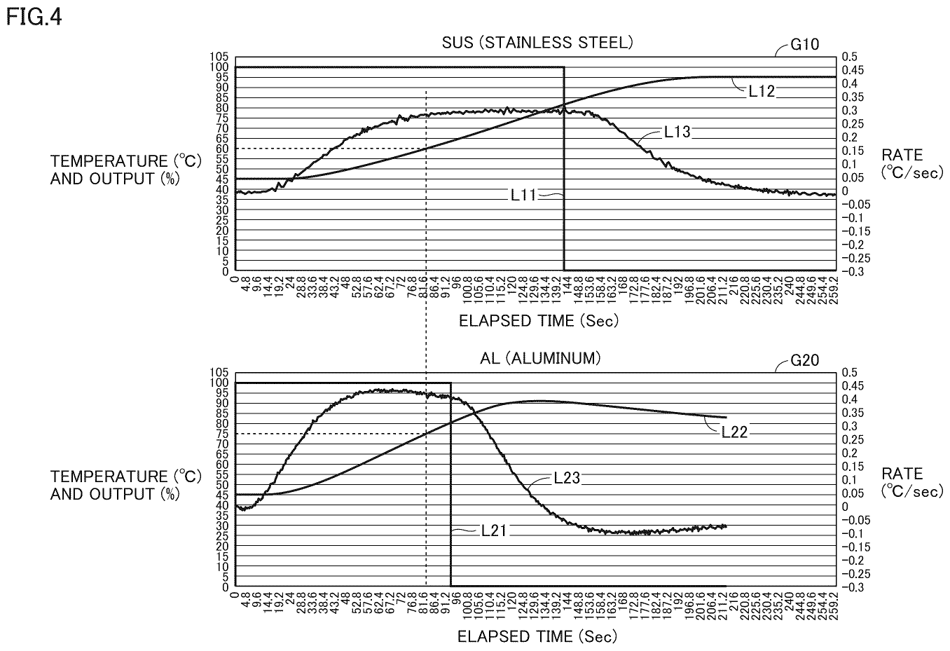

FIG. 4 is a diagram showing the information shown in FIG. 3 with different annotations from FIG. 3. In FIG. 4, the dashed line indicates the timing at which the rate of the measurement result in line L13 of graph G10 reaches the maximum value (maximum heating rate (0.3° C./sec)). The dashed line further indicates the same elapsed time in graph G20 as the said timing.

At the said timing, the measurement result of the temperature sensor 79 is about 60° C. as shown by line L12, and the temperature measured by the additional sensor is about 75° C. as shown by line L22. This means that when the maximum heating rate appears in the measurement result of the temperature sensor 79, there is a temperature difference of 15° C. or more between the first member and the second member.

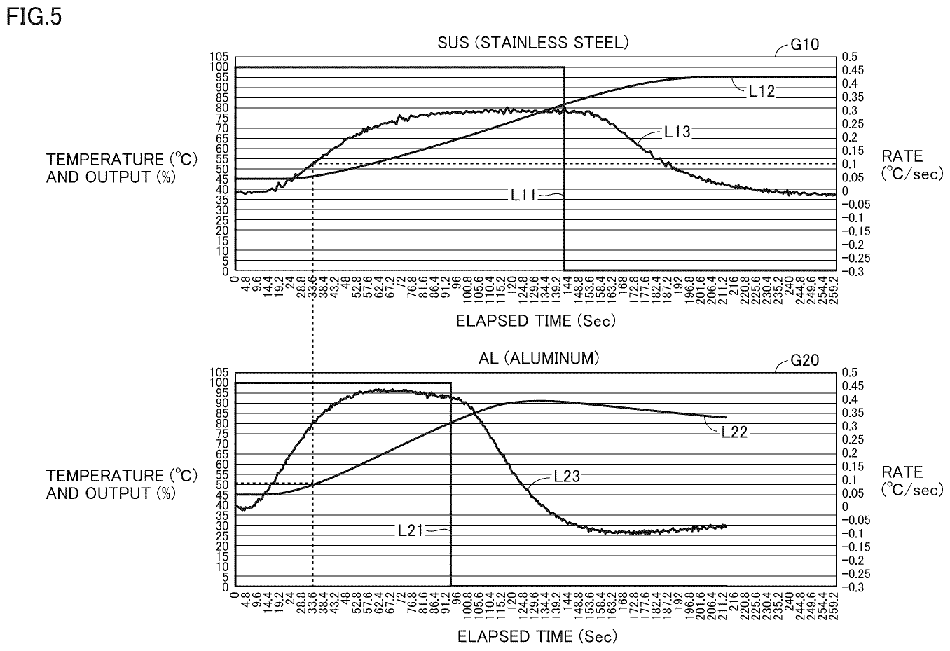

FIG. 5 is a diagram showing the information shown in FIG. 3 with different annotations from FIG. 3. In FIG. 5, the dashed line indicates the timing at which the heating rate reaches 0.1° C./sec in line L13 of graph G10. The dashed line further indicates the same elapsed time in graph G20 as the said timing.

0.1° C./sec is an example of a heating rate set based on the maximum heating rate. More specifically, if the heating rate exceeds the maximum heating rate, the temperature difference between the first member and the second member cannot be determined. Therefore, the temperature between the first member and the second member can be specified based on the timing when the heating rate reaches a given value lower than the maximum heating rate. As the heating rate to be used, for example, a value of about ⅓ of the maximum heating rate is used.

At the timing shown in FIG. 5, the measurement result of the temperature sensor 79 is about 46° C. as shown by line L12, and the temperature measured by the additional sensor is about 51° C. as shown by line L22. This means that when the heating rate in the measurement result of the temperature sensor 79 is 0.1° C./sec, the temperature difference between the first member and the second member is about 5° C.

### Determination of Start Threshold for Output Reduction Control

As explained with reference to FIG. 3, the time required from the start of heating to the rise in the first member (rise time) is 14 seconds. This means that a delay of 14 seconds occurs until the first member reaches the target temperature from the start of heating. Also, as explained with reference to FIG. 3, after the output of the heating device 70H is switched off, the measurement result of the temperature sensor 79 increases by 15° C.

Further, as explained with reference to FIG. 4, the maximum value (maximum heating rate) in the measurement result of the temperature sensor 79 is 0.3° C./sec, and there is a temperature difference of 15° C. or more between the first member and the second member when the maximum heating rate appears.

Furthermore, as explained with reference to FIG. 5, when the heating rate is 0.1° C./sec, the temperature difference between the first member and the second member is about 5° C.

From the above, when the heating rate is 0.1° C./sec, even if the above-mentioned delay (14 seconds) occurs, the temperature increase is suppressed to a maximum of 1.4° C. This temperature is smaller than the temperature difference (about 5° C.) between the first member and the second member when the heating rate is 0.1° C./sec. Therefore, by changing the control from the basic control to the output reduction control on the condition that the heating rate in the measurement result of the temperature sensor 79 is 0.1° C./sec or more, overshoot in the first member can be avoided. In other words, the heating rate being 0.1° C./sec or more is an example of a disengagement condition (a condition for disengaging the control of the heating device 70H from the basic control and transitioning to the output reduction control) being met. In this sense, “0.1° C./sec” can be an example of a threshold (start threshold) for switching the control from the basic control to the output reduction control.

The fact that the heating rate in the measurement result of the temperature sensor 79 is 0.1° C./sec or more constitutes an example of the temperature of the first member being outside a controllable range with respect to the temperature of the second member (i.e., outside the first range). More specifically, after reaching the maximum heating rate (0.3° C./sec), the heating rate hardly increases, but the difference between the first temperature and the second temperature tends to widen. Unless the heating rate is equal to or less than the maximum heating rate, it cannot be said that the temperature difference is within a controllable range. In this sense, it should be determined that the temperature of the first member is outside the controllable range with respect to the temperature of the second member on the condition that the heating rate in the measurement result of the temperature sensor 79 exceeds the maximum heating rate. In the present embodiment, in consideration of individual differences among the components of the gas chromatograph 1, 0.1° C., which is slightly smaller than 0.3° C., is adopted as the threshold in order to reliably specify that the temperature difference is within the controllable range.

Note that when the above-mentioned additional sensor is provided in the gas chromatograph 1, as another example of the start threshold, the difference between the direct measurement results of the respective temperatures of the first member and the second member may be adopted. More specifically, the control may be switched from the basic control to the output reduction control on the condition that this temperature difference is equal to or greater than a given value (for example, about 5° C.) . In this case, the temperature difference being equal to or greater than the said given value is another example of the disengagement condition being met.

### Determination of Release Threshold for Output Reduction Control

When the heating rate in the measurement result of the temperature sensor 79 is less than 0.1° C./sec, the control is changed from the output reduction control to the basic control. The fact that the heating rate in the measurement result of the temperature sensor 79 is less than 0.1° C./sec is an example of a return condition (a condition for returning the control of the heating device 70H from the output reduction control to the basic control). By returning the control of the heating device 70H to the basic control in response to the satisfaction of the return condition, the output of the heating device 70H can be controlled to stabilize the temperature of the first member near the target temperature earlier in a state where the possibility of overshoot occurring in the first member is low.

The fact that the heating rate in the measurement result of the temperature sensor 79 is less than 0.1° C./sec constitutes an example of the temperature of the first member being within the second range with respect to the temperature of the second member. In this case, “0.1° C./sec” constitutes an example of a threshold (release threshold) for switching the control from the output reduction control to the basic control.

The release threshold may be the same value as the above-mentioned start threshold. In this case, the second range means the same range as the first range.

Note that the release threshold may have a slightly larger value than the start threshold. More specifically, a value larger than the start threshold by about 0.5° C. may be set as the release threshold so that chattering does not occur even if the actual measured value of the temperature sensor 79 fluctuates instantaneously due to disturbance and/or noise. For example, if the start threshold is 0.10° C./sec, the release threshold may be 0.15° C./sec.

Note that when the above-mentioned additional sensor is provided in the gas chromatograph 1, as another example of the release threshold, the difference between the direct measurement results of the respective temperatures of the first member and the second member may be adopted. More specifically, the control may be switched from the output reduction control to the basic control on the condition that this temperature difference is less than a given value (for example, about 5° C.). In other words, this temperature difference being less than the given value is another example of the return condition being met.

### Processing Flow

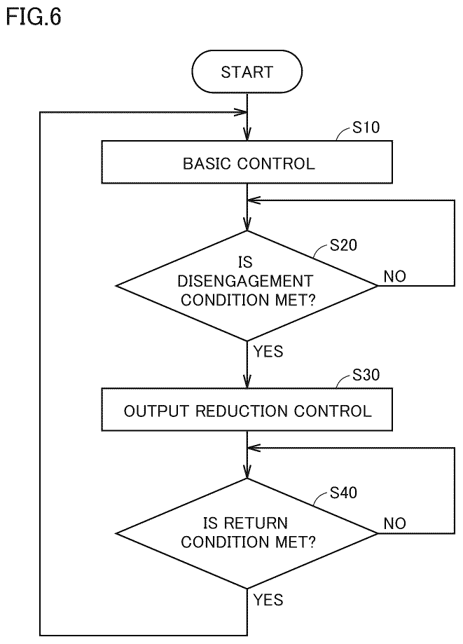

FIG. 6 is a flowchart of an output control process of the heating device 70H for controlling the first conduit 71 housing the filament F to a target temperature. In one implementation, the process of FIG. 6 is executed by the CPU of the control unit 80 executing a given program. In other words, an example of the “controller” in the present embodiment is realized by the control unit 80 executing the said given program. In one implementation, the control unit 80 starts the process of FIG. 6 in response to being instructed to start the temperature control of the first conduit 71. The content of the process will be described below with reference to FIG. 6.

In step S10, the control unit 80 starts the basic control for the heating device 70H. The control in step S10 corresponds to the function as the FB control unit 200 (FIG. 2).

In step S20, the control unit 80 determines whether the above-mentioned “disengagement condition” has been met. The control unit 80 repeats the control of step S20 until it determines that the “disengagement condition” has been met (NO in step S20), and when it determines that the “disengagement condition” has been met (YES in step S20), it proceeds to step S30. The control in step S20 corresponds to the function as the monitoring unit 201 (FIG. 2).

In step S30, the control unit 80 switches the control for the heating device 70H from the basic control to the output reduction control. The control in step S30 corresponds to the function as the output limiting unit 202 (FIG. 2).

In step S40, the control unit 80 determines whether the above-mentioned “return condition” has been met. The control unit 80 repeats the control of step S40 until it determines that the “return condition” has been met (NO in step S40), and when it determines that the “return condition” has been met (YES in step S40), it returns the control to step S10. Thereby, the control for the heating device 70H is returned to the basic control. The control in step S40 corresponds to the function as the monitoring unit 201 (FIG. 2).

When starting the process of FIG. 6, the control unit 80 may execute a control to set the output of the heating device 70H to 100% before the basic control of step S10. In one implementation, the control unit 80 may execute a control to set the output of the heating device 70H to 100% instead of PID control during a period when the temperature difference between the first member and the second member is considered to be 20° C. or more, and when it determines that such a period has ended, it may execute the heating control of step S10.

When the temperature difference between the first member and the second member is 20° C. or more, the proportional term in the PID control becomes 100% or more. The range where the proportional term is 100% or less is called the “proportional band”. Outside the “proportional band”, the output of the heating device 70H is controlled to 100% in order to rapidly raise the temperature of the second member. It is considered that the possibility of overshoot is low even if the basic control of the output of the heating device 70H is performed when the difference between the measurement result of the temperature sensor 79 and the temperature measured by the additional sensor becomes 15° C. or more (that is, when the measurement result of the temperature sensor 79 is 15° C. or more below the target temperature). The “20° C.” is adopted as an example of a value equal to or greater than the said difference.

In this specification, the control of the heating device of a thermal conductivity detector has been described with an example where the thermal conductivity detector is mounted on a gas chromatograph. However, the control of the heating device of the thermal conductivity detector described in this specification is not limited to the case where the thermal conductivity detector is mounted on a gas chromatograph, and can be applied to the control of the heating device of a thermal conductivity detector in any case.

### Aspects

It is understood by those skilled in the art that the plurality of exemplary embodiments described above are specific examples of the following aspects.
(Item 1) A thermal conductivity detection unit according to one aspect may include a thermal conductivity detector and a controller configured to control the thermal conductivity detector, wherein the thermal conductivity detector includes a first member, a second member having a higher thermal conductivity than the first member, a heating device housed in the second member, and a filament and a temperature sensor housed in the first member, and the controller is configured to control an output of the heating device with an output corresponding to a measurement result of the temperature sensor in a basic control, and when a temperature of the first member is outside a first range with respect to a temperature of the second member, control the output of the heating device with an output corresponding to the measurement result in an output reduction control, and in the output reduction control, the output of the heating device is controlled with an output that is reduced from the output corresponding to the measurement result in the basic control.

According to the thermal conductivity detection unit described in Item 1, a technology is provided for improving the accuracy of operation control of a heating device of a thermal conductivity detector.
(Item 2) In the thermal conductivity detection unit described in Item 1, the controller may determine that the temperature of the first member is outside the first range with respect to the temperature of the second member when a temperature increase value per unit time in the measurement result is equal to or greater than a predetermined heating rate.

According to the thermal conductivity detection unit described in Item 2, a temperature sensor for measuring the temperature of the second member is not required, and the manufacturing cost of the thermal conductivity detection unit can be reduced.
(Item 3) In the thermal conductivity detection unit described in Item 1 or 2, the controller may be configured to, after the output reduction control, control the output of the heating device with the output corresponding to the measurement result in the basic control when the temperature of the first member is within a second range with respect to the temperature of the second member.

According to the thermal conductivity detection unit described in Item 3, the operation of the heating device is controlled to make the temperature of the filament reach a target temperature earlier while suppressing the occurrence of overshoot.
(Item 4) In the thermal conductivity detection unit described in Item 3, the controller may determine that the temperature of the first member is within the second range with respect to the temperature of the second member when a difference between the measurement result and a target temperature in the control of the output of the heating device is equal to or less than a predetermined temperature.

According to the thermal conductivity detection unit described in Item 4, a temperature sensor for measuring the temperature of the second member is not required, and the manufacturing cost of the thermal conductivity detection unit can be reduced.
(Item 5) In the thermal conductivity detection unit described in any one of Items 1 to 4, the basic control may be PID control.

According to the thermal conductivity detection unit described in Item 5, the operation of the heating device can be controlled with high accuracy with respect to the target temperature.
(Item 6) In the thermal conductivity detection unit described in any one of Items 1 to 5, the output reduction control may include turning off the output of the heating device.

According to the thermal conductivity detection unit described in Item 6, overshoot in the first member can be more reliably avoided.
(Item 7) In the thermal conductivity detection unit described in any one of Items 1 to 6, the output reduction control may include calculating a value obtained by subtracting a constant value from the output corresponding to the measurement result in the basic control as the output of the heating device.

According to the thermal conductivity detection unit described in Item 7, overshoot in the first member is avoided, and the temperature of the first member can be stabilized near the target temperature early.
(Item 8) In the thermal conductivity detection unit described in any one of Items 1 to 7, the first member may include stainless steel, and the second member may include aluminum.

According to the thermal conductivity detection unit described in Item 8, heat from the heating device is easily propagated to other elements, and the temperature near the filament is stabilized.
(Item 9) A gas chromatograph according to one aspect may include a sample vaporization unit that generates a sample gas by vaporizing a sample, a column that separates components of the sample gas generated by the sample vaporization unit, and the thermal conductivity detection unit described in any one of Items 1 to 8, wherein the thermal conductivity detection unit detects the thermal conductivity of the sample gas for each component separated by the column.

According to the gas chromatograph described in Item 9, a technology is provided for improving the accuracy of operation control of a heating device of a thermal conductivity detector.

The embodiments disclosed this time should be considered as illustrative in all respects and not restrictive. The scope of the present disclosure is indicated by the claims rather than by the description of the embodiments above, and all changes that come within the meaning and range of equivalency of the claims are intended to be embraced therein. Further, it is intended that each technology in the embodiments can be implemented alone or in combination with other technologies in the embodiments as much as possible, as necessary.

### Reference Signs List

1 Gas chromatograph, 10 Gas tank, 20, 50 Flow rate adjustment unit, 30 Sample vaporization unit, 40 Column, 60 Switching valve, 70 Thermal conductivity detector, 70A Aluminum block, 70H Heating device, 70X Cell block, 71 First conduit, 75, 77 Carrier gas introduction conduit, 76 Sample introduction conduit, 78 Exhaust conduit, 78e Exhaust port, 79 Temperature sensor, 80 Control unit, 200 FB control unit, 201 Monitoring unit, 202 Output limiting unit, 220 Controlled object, F Filament, G10, G20 Graph, L11, L12, L13, L21, L22, L23 Line.

1. A thermal conductivity detection unit, comprising:
a thermal conductivity detector; and
a controller configured to control the thermal conductivity detector,
wherein the thermal conductivity detector includes:
a first member;
a second member having a higher thermal conductivity than the first member;
a heating device housed in the second member; and
a filament and a temperature sensor housed in the first member,
the controller is configured to:
control an output of the heating device with an output corresponding to a measurement result of the temperature sensor in a basic control, and
when a temperature of the first member is outside a first range with respect to a temperature of the second member, control the output of the heating device with an output corresponding to the measurement result in an output reduction control, and
in the output reduction control, the output of the heating device is controlled with an output that is reduced from the output corresponding to the measurement result in the basic control.

2. The thermal conductivity detection unit according to claim 1, wherein the controller determines that the temperature of the first member is outside the first range with respect to the temperature of the second member when a temperature increase value per unit time in the measurement result is equal to or greater than a predetermined heating rate.

3. The thermal conductivity detection unit according to claim 1, wherein the controller is configured to, after the output reduction control, control the output of the heating device with the output corresponding to the measurement result in the basic control when the temperature of the first member is within a second range with respect to the temperature of the second member.

4. The thermal conductivity detection unit according to claim 3, wherein the controller determines that the temperature of the first member is within the second range with respect to the temperature of the second member when a difference between the measurement result and a target temperature in the control of the output of the heating device is equal to or less than a predetermined temperature.

5. The thermal conductivity detection unit according to claim 1, wherein the basic control is PID control.

6. The thermal conductivity detection unit according to claim 1, wherein the output reduction control includes turning off the output of the heating device.

7. The thermal conductivity detection unit according to claim 1, wherein the output reduction control includes calculating, as the output of the heating device, a value obtained by subtracting a constant value from the output corresponding to the measurement result in the basic control.

8. The thermal conductivity detection unit according to claim 1, wherein
the first member includes stainless steel, and
the second member includes aluminum.

9. A gas chromatograph, comprising:
a sample vaporization unit that generates a sample gas by vaporizing a sample;
a column that separates components of the sample gas generated by the sample vaporization unit; and
the thermal conductivity detection unit according to claim 1,
wherein the thermal conductivity detection unit detects the thermal conductivity of the sample gas for each component separated by the column.
